(19) United States
(12) Patent Application Publication
(10) Pub. No.: US 2002/0193440 A1
(43) Pub. Date: Dec. 19, 2002

(54) Liothyronine sodium and levothyroxine sodium combination

(76) Inventors: Steven C. Ritter, Canton, MI (US); Steven E. Springer-Wilson, Dearborn, MI (US); Robert Gault, Grosse Pointe Woods, MI (US)

Correspondence Address: John Conway, BASF Bioresearch Corporation, 100 Research Drive, Worcester, MA, 01605, US

(21) Appl. No.: 10/079,375

(22) Filed: Feb. 20, 2002

Related U.S. Application Data
(60) Provisional application No. 60/269,992, filed on Feb. 20, 2001.

(51) Int. Cl. A61K 31/198

(52) U.S. Cl. 514/567

(57) ABSTRACT

Novel formulations useful for thyroid hormone replacement and suppression therapy are described herein. While dosage formulations containing iodinated thyronines, levothyroxine sodium (T4) or liothyronine sodium (T3), as the active pharmaceutical ingredient (API) dispersed in a polymeric solid are known, manufacturing such formulations with acceptable dose content uniformity is difficult due to the high biological activity of T4 and T3. Described herein is the preparation of a single API formulation containing both T4 and T3 in a pharmaceutically effective ratio, wherein the dose content uniformity is the same for T3 as for T4 even if the T3 dose is extremely low. The present invention provides a method for the preparation of an API wherein T3 is uniformly distributed within the crystal lattice of T4 to form a solid solution by cooling suitable solutions of levothyroxine sodium and liothyronine sodium, a pharmaceutical composition containing such an API, and a method to use such an API in the treatment of thyroactive disorders.

### CROSS-REFERENCE TO RELATED APPLICATION

This application claims priority to U.S. provisional application No. 60/269,992, filed Feb. 20, 2001.

### FIELD OF THE INVENTION

This invention relates to preparations useful in replacement therapy for thyroactive material normally supplied by the thyroid gland.

### BACKGROUND OF THE INVENTION

The thyroid gland releases various iodinated thyronines and by doing so modulates a body's energy metabolism. Two of the iodinated thyronines are levothyroxine and liothyronine. Dosage forms containing as the active pharmaceutical ingredient (API) either levothyroxine sodium (T4) or liothyronine sodium (T3) are widely used for thyroid hormone replacement and suppression therapy. The usefulness of a dosage form containing both APIs is recognized, and tablets with a T4/T3 molar ratio of 3.37:1 (weight ratio 4:1) are marketed under the generic name liotrix.

Because of the high biological activity of T4 and T3, the typical dose of each is measured in micrograms, and a T3 dose as low as 1 μg may be required when the T4/T3 molar ratio is high. Manufacturing dosage forms with acceptable dose content uniformity can be difficult when the dose is so low.

U.S. Pat. No. 5,324,522 discloses dosage forms containing T4 and T3, wherein either T3 or both T3 and T4 are in a sustained or prolonged release form. The disclosed dosage forms have a T4/T3 molar ratio ranging from 1:1 to 50:1. The patent also describes preparation of such a dosage form by combining T4 and T3 with inert excipients. The lowest dosage content of T3 is 3 micrograms.

U.S. Pat. No. 3,477,954 describes preparation of a combination of levothyroxine and liothyronine as the free acids rather than the sodium salts, with a T4/T3 molar ratio of approximately 2.9-6.7:1 (weight ratio as neutral amino acids 3.5-8.0:1). The levothyroxine-liothyronine mixture is prepared in a two-step process by treatment of levothyroxine with a tertiary phosphine in a dipolar aprotic solvent, followed by hydrolysis of the reaction product. The levothyroxine/liothyronine ratio is controlled by the ratio of levothyroxine to phosphine used. No procedure is given for conversion of the levothyroxine-liothyronine mixture to a mixture of levothyroxine sodium and liothyronine sodium, which are the preferred forms for dosage form manufacture.

Accordingly, a need exists in the art for an improved method for the preparation of low dosage forms of T3 of acceptable uniformity.

### SUMMARY OF THE INVENTION

Surprisingly, it has now been found that this difficulty can be greatly reduced by the use of a single API containing both T4 and T3 in the desired ratio. When this single API is combined with excipients to manufacture a dosage form, the dose content uniformity is the same for T3 as for T4 even if the T3 dose is extremely low. Use of such a combination API is advantageous only if the T4 and T3 are intimately and homogeneously mixed so that they could not separate and become differently distributed during dosage form manufacture. It has now been found that such an API can be produced.

The present invention provides a method for the preparation of an API wherein T3 is uniformly distributed within the crystal lattice of T4 to form a solid solution, a pharmaceutical composition containing such an API, and a method to use such an API in the treatment of thyroactive disorders.

### DETAILED DESCRIPTION OF THE INVENTION

Surprisingly it has now been found that the crystals produced by cooling certain suitable solutions of levothyroxine sodium and liothyronine sodium consist of an intimate homogeneous mixture of levothyroxine sodium and liothyronine sodium. T3 molecules are uniformly distributed in the T4 crystal lattice to form a solid solution, in the sense familiar to crystallographers, not the sense used in the pharmaceutical industry to describe a formulation in which an API is dispersed in a polymeric solid. The T4/T3 ratio in the product can be controlled by suitably altering the quantities of T4 and T3 in the solution from which the product is crystallized.

In one embodiment of the invention, the T3/T4 solid solution of the invention is formed from the solution of levothyroxine sodium and liothyronine sodium.

In another embodiment of the invention, the T3/T4 solid solution of the invention is formed from the solution of other forms of levothyroxine and liothyronine with the addition of external sources of sodium.

More particularly, this invention relates to a solid solution of levothyroxine sodium and liothyronine sodium wherein molecules of liothyronine sodium are contained within the crystal lattice of levothyroxine sodium crystals. The molar ratio of levothyroxine sodium to liothyronine in the solid solution is from about 5 to about 100, preferably from about 10 to about 50, more preferably from about 15 to about 20, most preferably from about 16 to about 18. The percentage of liothyronine present in the API of the invention, expressed on an anhydrous basis, is from about 0.84 wt % to about 14.4 wt % of the product, more preferably from about 1.7 wt % to about 7.8 wt %, even more preferably from about 4.0 wt % to about 5.3 wt %, and most preferably from about 4.5 wt % to about 5.0 wt %.

Although levothyroxine sodium and liothyronine sodium must be present in the solution from which the T4-T3 combination is to be crystallized, the solution may be prepared by combining other forms of levothyroxine and liothyronine with a suitable source of sodium. Either levothyroxine or liothyronine or both can be added to the solution as the neutral amino acid or as an acid salt such as the hydrochloride. For example, a mixture of levothyroxine and liothyronine prepared according to U.S. Pat. No. 3,477,954 could be used for part or all of the levothyroxine and liothyronine. As another example, a T4/T3 combination prepared as described herein could be used as part or all of the starting material for a subsequent preparation, with the quantities of other raw materials adjusted to increase or decrease the T4/T3 ratio as desired.

More particularly, the process of the invention relates to a process for the preparation of a solid solution of levothyroxine sodium and liothyronine sodium wherein molecules of liothyronine sodium are contained within the crystal lattice structure of levothyroxine sodium which comprises

a. dissolving a mixture of levothyroxine and liothyronine in a solvent mixture of water, an alcohol and sodium carbonate at a temperature between about room temperature and 100° C.;

b. cooling the mixture; and

c. obtaining the resulting product.

The crystallization is carried out in a mixture of solvents such as the mixture of water and an alcohol. The preferred solvent mixture is a mixture of water, n-propanol, and sodium carbonate. The relative amounts of water and n-propanol can also be used to control the T4/T3 ratio in the product. The percentage by weight of n-propanol in the water/n-propanol mixture is from about 5% to about 20%.

The T4/T3 ratio in the product is not the ratio of T4/T3 that is mixed together, but a linear relationship between mole ratio T4/T3 charged and mole ratio T4/T3 produced permits choice of the charge ratio required to produce a particular target. This linear relationship depends on the solvent mixture, and a plot can be generated for each solvent mixture.

The determination of the ratio of T3 to T4 in the crystallized product is carried out by assays known in the art. More preferably, the T4 and T3 assays of the product are conveniently determined by liquid chromatography using compendial methods already in place for levothyroxine sodium and liothyronine sodium APIs, and the T4/T3 ratio can then be calculated from the assays.

The T4/T3 ratio thus determined for the product depends on the quantities of T4, T3, and other components present in the solution from which the product was crystallized, but it is not sensitive to crystallization temperature. Thus, product crystallized from a given solution at 20° C. has the same T4/T3 ratio as product crystallized by further cooling the solution to 10° C., as is illustrated in Example 3. This is advantageous in making the process robust.

The ratio of T4 to T3 in the final product can be altered during the crystallization process by the addition of additional T3 or T4 as is deemed necessary to obtain the desired final ratio.

The T4/T3 weight ratio is independent of product particle size. This can be demonstrated by sieving the product into various size fractions and determining the T4/T3 ratio for each, as was done in Example 3.

Pure T4 crystallizes as a pentahydrate containing 10.13% water by weight. It is not surprising, then, that the T4/T3 combinations whose preparation is described here contain up to 10% water incorporated into the crystals. The water content is conveniently determined by the compendial method for water in levothyroxine sodium, loss on drying (LOD), or by Karl Fischer titration.

The API of this invention can then be formulated into pharmaceutical dosage forms as is known in the art. More particularly, the API can be substituted for T4 or for T3 in a known pharmaceutical composition. This produces a dosage form having a T3/T4 combination that can be produced uniformly and reproducibly.

Additional embodiments and non-limiting descriptions are set forth below in the Examples.

### EXAMPLE 1

A mixture of levothyroxine sodium (40.0 g), liothyronine (8.32 g), sodium carbonate monohydrate (21.2 g), n-propanol (102 g), and water (580 g) was heated to 55° C. in a jacketed 3 L cylindrical reaction flask equipped with a turbine agitator. When the solids had dissolved, the solution was cooled at 0.5° C. per minute to 10°. The product slurry was held at 10° C. for one hour and then filtered. The solid was washed with 95% ethanol (3×24 g) and then allowed to dry at ambient temperature and pressure. 31.5 g off-white crystals were obtained. Liquid chromatographic analysis showed 5.6 wt % liothyronine sodium, corresponding to a T4/T3 molar ratio of 17.0.

Photomicrographs of the product crystals were indistinguishable from photomicrographs of levothyroxine sodium crystallized under the same conditions without added liothyronine sodium. The absence of crystals of a different morphology is consistent with uniform distribution of T3 within the T4 crystal lattice, although it does not prove uniformity.

An energy-dispersive image for iodine was obtained using an Amray 1830 scanning electron microscope equipped with an EDAX Phoenix energy dispersive spectrometer. The image was indistinguishable from that obtained using all electron energies, indicating no variation of iodine concentration across the surface of the crystals. This result, too, is consistent with uniform distribution of T3 within the T4 crystal lattice, although it again does not prove uniformity.

An X-ray powder diffraction pattern of the product, obtained at a synchrotron facility to permit sensitive detection of T3 lines, was identical to that of levothyroxine sodium prepared without addition of T3. In contrast, a physical mixture of T4 and T3 containing 6% T3 clearly showed a line characteristic of T3. The absence of the T3 line in the XRPD pattern of the T4/T3 combination prepared by crystallization proves that the crystallization product is a solid solution of T3 in T4 rather than a mixture of separate T4 and T3 crystals.

### EXAMPLE 2

A mixture of levothyroxine sodium (1.00, 1.50 or 2.00 g), liothyronine (0.13-0.31 g), sodium carbonate monohydrate (0.53 g), n-propanol (2.56 g), and water (14.6 g) was heated to 60° C. to dissolve the solids. The solution was cooled in an ice bath for one hour, and the resulting product slurry was filtered. The solid was washed with 95% ethanol (1 mL) and allowed to dry at ambient temperature and pressure. The T3 content of the product, determined by liquid chromatography, ranged from 2.4-7.5% (T4/T3 molar ratio ranging from 9.1 to 31) depending on the weights of T4 and T3 charged:

| | 1.00 g | 1.50 g. | 2.00 g |
|---|---|---|---|
| g T3 | T4 | T4 | T4 |
| 0.13 | 3.4 | 3.0 | 2.4 |
| 0.18 | 4.7 | 4.0 | 3.2 |
| 0.26 | 6.1 | 6.2 | 4.3 |
| 0.31 | 7.5 | 6.0 | 5.0 |

### EXAMPLE 3-7

A mixture of levothyroxine sodium (LOD 9.1%, 80.0 g), liothyronine sodium (LOD 0.7%, 16.46 g), sodium carbonate monohydrate (42.90 g), n-propanol (207 g), and water (1174 g) was heated to 55° C. in a jacketed 3-L cylindrical reaction flask equipped with a turbine agitator. When the solids had dissolved, the solution was cooled at 0.5° C. per minute to 10°. Solid began to crystallize at approximately 28° C. The product slurry was held at 10° C. for one hour and then filtered. The solid was washed with 95% ethanol (3×49 g) and then allowed to dry at ambient temperature and pressure. 59.7 g off-white crystals were obtained.

The product contained 9.1% water by LOD. Liquid chromatographic analysis showed 4.8 wt % liothyronine sodium, corresponding to a T4/T3 molar ratio of 19.9.

Photomicrographs of the product crystals were indistinguishable from photomicrographs of levothyroxine sodium crystallized under the same conditions without added liothyronine sodium.

Aliquots collected during cooling at temperatures of 25°, 20°, and 15°were filtered, and the solid was washed with 95% ethanol and allowed to dry at room temperature. Liquid chromatographic analysis of the three products for T3 showed 5.0%, 4.7%, and 4.7%, respectively.

Two of the products prepared in this series were combined and sieved using 8-inch 100-, 170-, and 270-mesh sieves and a sieve shaker. The four sieve cuts were weighed, and the T3 content of each sieve cut was determined by liquid chromatography.

| size | | area % |
|---|---|---|
| (μm) | wt % | T3 |
| >150 | 4.5 | 4.87 |
| >90 | 27.3 | 4.77 |
| >53 | 25.4 | 4.64 |
| <53 | 32.8 | 4.55 |

Four additional preparations by the same procedure illustrate the reproducibility of the product characteristics:

| Example | product | LOD | T3 |
|---|---|---|---|
| No. | wt (g) | (%) | (%) |
| 3 | 59.7 | 9.1 | 4.8 |
| 4 | 60.9 | 9.1 | 4.7 |
| 5 | 62.0 | 8.8 | 4.7 |
| 6 | 60.8 | 8.5 | 4.7 |
| 7 | 59.7 | 8.5 | 4.7 |

### EXAMPLE 8

A mixture of levothyroxine sodium (LOD 9.8%, 40.0 g), liothyronine sodium (LOD 0.7%, 8.17 g), sodium carbonate monohydrate (21.27 g), n-propanol (43 mL), and water (650 mL) was heated to 75° C. in a jacketed 3-L cylindrical reaction flask equipped with a turbine agitator. When the solids had dissolved, the solution was cooled at 0.5° C. per minute to 10°. The product slurry was held at 10° C. for one hour and then filtered. The solid was washed with 95% ethanol (3×30 mL) and then allowed to dry at ambient temperature and pressure. 35.8 g off-white crystals were obtained.

The product contained 8.6% water by LOD. Liquid chromatographic analysis showed 3.5 wt % liothyronine sodium, corresponding to a T4/T3 molar ratio of 27.5. Photomicrographs of the product crystals were indistinguishable from photomicrographs of levothyroxine sodium crystallized under the same conditions without added liothyronine sodium.

1. A solid solution of levothyroxine sodium and liothyronine sodium wherein molecules of liothyronine sodium are contained within the crystal lattice of levothyroxine sodium crystals.

2. The solid solution of claim 1 wherein the molar ratio of levothyroxine sodium to liothyronine is from about 5 to about 100.

3. The solid solution of claim 2 wherein the molar ratio of levothyroxine sodium to liothyronine is from about 10 to about 50.

4. The solid solution of claim 3 wherein the molar ratio of levothyroxine sodium to liothyronine is from about 15 to about 20.

5. The solid solution of claim 4 wherein the molar ratio of levothyroxine sodium to liothyronine is from about 16 to about 18.

6. The solid solution of claim 1 wherein liothyronine sodium is from about 0.84 wt % to about 14.4 wt % of the product on an anhydrous basis.

7. The solid solution of claim 6 wherein liothyronine sodium is from about 1.7 wt % to about 7.8 wt % of the product.

8. The solid solution of claim 7 wherein liothyronine sodium is from about 4.0 wt % to about 5.3 wt % of the product.

9. The solid solution of claim 8 wherein liothyronine sodium is from about 4.5 wt % to about 5.0 wt % of the product.

10. A process for the preparation of a solid solution of levothyroxine sodium and liothyronine sodium wherein molecules of liothyronine sodium are contained within the crystal lattice structure of levothyroxine sodium which comprises
d. dissolving a mixture of levothyroxine and liothyronine in a solvent mixture of water, an alcohol and sodium carbonate at a temperature between about room temperature and 100° C.;
e. cooling the mixture; and
f. obtaining the resulting product.

11. The process of claim 10 wherein the alcohol is n-propanol.

12. The process of claim 11 wherein the percentage by weight of n-propanol in the water/n-propanol mixture is from about 5% to about 20%.

13. The process of claim 10 wherein at least one of levothyroxine and liothyronine is in a non-sodium salt form.

14. The process of claim 13 wherein the alcohol is n-propanol.

15. The process of claim 14 wherein the percentage by weight of n-propanol in the water/n-propanol mixture is from about 5% to about 20%.

16. The process of claim 13 wherein liothyronine is in a non-sodium salt form.

17. The process of claim 16 wherein the alcohol is n-propanol.

18. The process of claim 17 wherein the percentage by weight of n-propanol in the water/n-propanol mixture is from about 5% to about 20%.

19. A method of treating thyroactive disorders in a patient which comprises administering a therapeutically active amount of the solid solution of claim 1.

20. A pharmceutical composition which comprises a pharmaceutically acceptable carrier and the solid solution of claim 1.
